(19) United States
(12) Patent Application Publication
(10) Pub. No.: US 2023/0204608 A1
(43) Pub. Date: Jun. 29, 2023

(54) WATER QUALITY MANAGEMENT METHOD, INFORMATION PROCESSING DEVICE, AND INFORMATION PROCESSING SYSTEM

(71) Applicant: ORGANO CORPORATION, Tokyo (JP)

(72) Inventors: Fumitaka ICHIHARA, Tokyo (JP); Tsukasa KONDO, Tokyo (JP); Hiroshi SUGAWARA, Tokyo (JP)

(73) Assignee: ORGANO CORPORATION, Tokyo (JP)

(21) Appl. No.: 17/915,762

(22) PCT Filed: Jan. 26, 2021
(86) PCT No.: PCT/JP2021/002600

§ 371 Date: Sep. 29, 2022

(30) Foreign Application Priority Data
Apr. 3, 2020 (JP) 2020-067756

(51) Int. Cl. G01N 35/00; G01N 1/10; G01N 15/06

(52) U.S. Cl. CPC G01N 35/00613; G01N 35/00732; G01N 1/10; G01N 35/0092; G01N 15/0618; G01N 2035/00475; G01N 2001/1025

(57) ABSTRACT

A water quality management method for performing at least one of a quantitative analysis and a qualitative analysis of fine particles contained in water to be analyzed that includes connecting a filtration device provided with a fine particle capturing membrane for capturing fine particles to a flow pipe through which the water to be analyzed flows, allowing the water to be analyzed to flow from the flow pipe and through the fine particle capturing membrane attached to the filtration device for a predetermined period of time to capture fine particles contained in the water to be analyzed to form a fine particle capturing membrane sample, and performing at least one of a quantitative analysis and a qualitative analysis of the fine particle capturing membrane sample of a target water flow period at an arbitrary timing.

### TECHNICAL FIELD

The present invention relates to a water quality management method for managing fine particle concentration in ultrapure water, and more particularly, to a water quality management method for quantifying an extremely small amount of fine particles present in ultrapure water, an information processing device used in the water quality management method, and an information processing system using the same.

### BACKGROUND OF ART

Ultrapure water is generally produced by treating water to be treated such as river water, groundwater and industrial water in a pretreatment step to obtain pretreated water, and then sequentially treating the pretreated water with a primary system pure water producing apparatus and a secondary system pure water producing apparatus (subsystem). This pretreatment step is a step of removing most of the suspended matter and organic matter in the water to be treated. The ultrapure water that is produced is supplied to, for example, a point of use for performing wafer cleaning or the like in a semiconductor device manufacturing factory. Ultrapure water is also widely used in pharmaceutical manufacturing processes, etc. The terms “pure water” and “ultrapure water” are not generally clearly defined. In this specification, high-purity water, which is generally described by terms such as “pure water,” “ultrapure water,” and the like, will be generically referred to as “ultrapure water”.

Ultrapure water has such high purity that the quantifying of impurities contained therein is also difficult. However, ultrapure water contains a small amount of impurities. The effect of ultra-trace components contained in ultrapure water on products such as semiconductor devices becomes non-negligible as the degree of integration in the device increases. For this reason, the need for ultrapure water having even higher purity than conventional ultrapure water has also been studied.

Ultrapure water produced by subsystems in semiconductor device manufacturing factories, etc. is supplied to the point of use through piping. The length of the pipe between the subsystem and the point of use may be as long as several hundred meters. Therefore, there are cases where impurities such as fine particles and metal ion components from the pipe are mixed into ultrapure water, although the quantity of such impurities is slight. In such cases, the characteristics of the semiconductor devices that are manufactured may be adversely affected. Particulates in particular, may cause defects such as pattern defects, disconnection, and dielectric strength reduction, raising the concern that yield will be directly affected. Therefore, strict control is required for both the particle size and the concentration of the fine particles. Recently, the necessity sometimes arises to control the concentration of fine particles to below a specified value. The same applies to ultrapure water used in the field of chemical production.

SEM (Scanning Electron Microscopy) method is known as a method for detecting fine particles in ultrapure water (e.g., see Non-Patent Document 1.) According to this method, pure water or ultrapure water is filtered using a filtration membrane, fine particles are captured on the filtration membrane, and the captured fine particles are detected using an optical microscope or a scanning electron microscope. As the filtration membrane, a filtration membrane is used that has a pore size smaller than the particle size of the particle to be detected. Thus, it is possible to detect particles having even smaller particle size. However, in order to ensure reliability of detection, it is desirable to capture a number of fine particles equal to or greater than the number of fine particles contained in the filtration membrane itself. To do so, a sufficient amount of pure water or ultrapure water must be passed through the filtration membrane. Further, the smaller the particle size of the particles to be detected, the smaller the pore size of the filtration membrane necessary to capture the fine particles, and this smaller pore size results in increased loss of pressure of the filtration membrane. As a result of these factors, long-time filtration is required to detect fine particles with small particle size.

In detecting fine particles using SEM method, a method of filtering pure water or ultrapure water using a centrifugal filter is known (e.g., see Patent Documents 1 and 2). Pure water or ultrapure water is pressurized by centrifugal force, and the flow rate of pure water or ultrapure water through the filtration membrane increases. Therefore, the time required for filtration is shortened.

### PRIOR ART DOCUMENTS

### Patent Document

[Patent Document 1] JP-4-136550A
[Patent Document 2] JP 2012-115810 A

### Non-Patent Document

[Non-Patent Document 1] Japanese Industrial Standards JIS K 0554-1995 “Methods for Measuring Fine Particles in Ultrapure Water”

### SUMMARY OF THE INVENTION

### Problem to be Solved by the Invention

SEM method described in Patent Document 1 and Patent Document 2 are improved methods for the purpose of measuring ultra-trace (minute) particles contained in ultrapure water. This SEM method also plays an important role for determining whether the water quality of ultrapure water meets specifications immediately after completion of the ultrapure water producing apparatus or after maintenance. Incidentally, when it is discovered that a product manufactured through the process of using ultrapure water has a defect or the like, it is necessary to analyze various factors that can be considered in order to identify the cause of the defect. As a part of the analysis, considering the possibility that there was an increase in the number of fine particles in the ultrapure water, fine particle analysis by SEM method of the ultrapure water used will be carried out. In this case, even if the fine particles contained in the ultrapure water were the cause of the defect, a considerable amount of time has usually elapsed from the occurrence of the event that caused the defect to the analysis. Therefore, the fine particles may already be absent in the ultrapure water at the investigation stage in which the analysis is carried out. This leaves the cause unclear despite the time and effort spent investigating.

It is an object of the present invention to provide a water quality management method that can perform analysis of a very small amount of fine particles in water that is analyzed after the event and that facilitates implementation of a defect analysis, an information processing device used in the water quality management method, and an information processing system using the same.

### A Means for Solving the Problem

The present invention is a water quality control method for quantitative analysis and/or qualitative analysis of fine particles contained in water to be analyzed, comprising:

a step of attaching a fine particle capturing membrane for capturing fine particles to a filtration device connected to a flow pipe through which the water to be analyzed flows,

a step of allowing the water to be analyzed to flow from the flow pipe over a predetermined period to the fine particle capturing membrane attached to the filtration device and capturing the fine particles contained in the water to be analyzed to form a fine particle capturing membrane sample, and

a step of performing at an arbitrary timing at least one of quantitative analysis and qualitative analysis of the fine particle capturing membrane sample of a water flow of a period of interest.

The present invention is an information processing device, comprising:

an input unit that receives input information based on an operation accepted from outside,

a database that stores period information, which indicates the time when a fine particle capturing membrane through which water to be analyzed flows for capturing fine particles of the water to be analyzed was attached to a flow pipe through which the water to be analyzed flowed in association with capturing membrane identification information uniquely conferred to the fine particle capturing membrane,

a retrieval unit that receives the capturing membrane identification information from the database based on date and time information included in input information the input unit receives, and

an output unit that supplies the capturing membrane identification information retrieved by the retrieval unit.

The present invention is an information processing system, comprising:

a filtration device,

an integrating flow meter,

an analyzer, and

an information processing device; wherein:

the filtration device comprises:

a fine particle capturing membrane that is removably provided from the filtration device and that captures fine particles of the water to be analyzed which flows through the fine particle capturing membrane,

the integrating flow meter is provided on the downstream side of the flow direction of the water to be analyzed of the filtration device and measures the integrated value of the water flow rate of the fine particle capturing membrane,

the information processing device comprises:

an input unit that receives input information based on an operation that is accepted from outside,

a database that stores period information that indicates the time when the fine particle capturing membrane was attached to the flow pipe through which the water to be analyzed flows in association with the capturing membrane identification information uniquely conferred to the fine particle capturing membrane,

a retrieval unit that retrieves the capturing membrane identification information from the database based on date and time information included in input information the input unit retrieves, and

an output unit that supplies the capturing membrane identification information retrieved by the retrieval unit,

wherein the analyzer performs at least one of a quantitative analysis and a qualitative analysis of the fine particle capturing membrane to which the capturing membrane identification information supplied by the output unit is conferred,

wherein the output unit supplies provided information based on the result of analysis performed by the analyzer.

### Advantageous Effects of the Invention

According to the present invention, it is possible to perform an analysis of a very small amount of fine particles in water to be analyzed after the event and to easily perform a defect analysis.

### BRIEF DESCRIPTION OF THE DRAWINGS

FIG. 1 is a diagram showing a filtration device of an embodiment of the present invention.

FIG. 2 is a flowchart for explaining the water quality management method.

FIG. 3 is a diagram illustrating an example of a connection location of the filtration device in a plant having a step of using ultrapure water.

FIG. 4 is a diagram showing a first example of an information processing system using the filtration device shown in FIG. 1.

FIG. 5 is a diagram showing an example of an internal configuration of the information processing device shown in FIG. 4.

FIG. 6 is a diagram showing an example of the association between the period information and the capturing membrane identification information stored in the database shown in FIG. 5.

FIG. 7 is a diagram showing an example of the association between the installation information and the capturing membrane identification information stored in the database shown in FIG. 5.

FIG. 8 is a flowchart for explaining an example of, among information processing methods, processing in the filtration device in the information processing system shown in FIG. 4.

FIG. 9 is a flowchart for explaining an example of, among information processing methods, the retrieval process in the information processing device in the information processing system shown in FIG. 4.

FIG. 10 is a diagram showing a second example of an information processing system using the filtration device shown in FIG. 1.

FIG. 11 is a diagram showing an example of the internal configuration of the information processing device shown in FIG. 10.

FIG. 12 is a sequence diagram for explaining an example of an information processing method in the information processing system shown in FIG. 10.

FIG. 13 is a flowchart for explaining an example of the details of the processing of Step S4 described with reference to the sequence diagram shown in FIG. 12.

FIG. 14 is a flowchart for explaining an example of details of the processing of Step S8 described with reference to the sequence diagram shown in FIG. 12.

### DESCRIPTION OF EMBODIMENTS

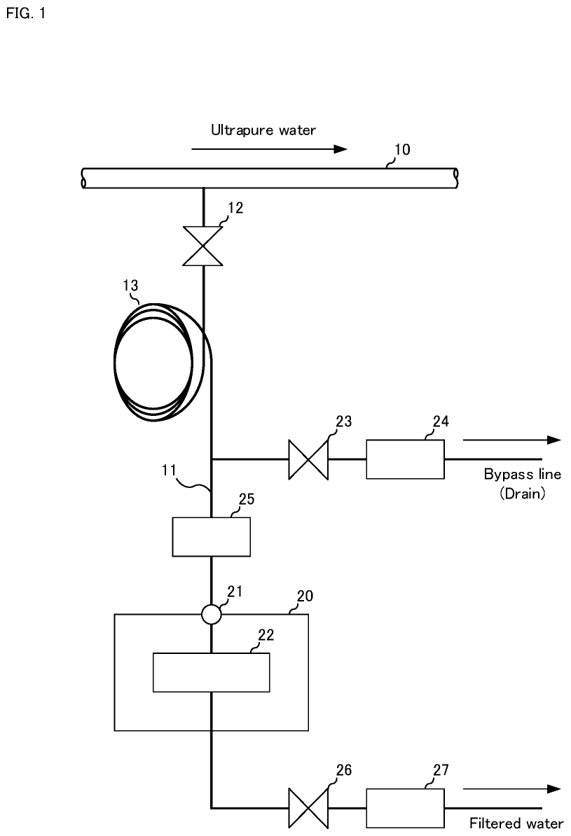

Hereinafter, preferred embodiments of the present invention will be described with reference to the accompanying drawings. FIG. 1 shows a filtration device of an embodiment of the present invention. Here, it is assumed that the water to be analyzed is ultrapure water that is used in the manufacturing process of products such as semiconductor devices and that is in contact with the products. The water to be analyzed to which the filtration device or the water quality management method of the present invention is applied is not limited thereto. Examples of the water to be analyzed include functional water, pure water (primary system), and chemical solutions such as IPA (isopropyl alcohol)

Flow pipe 11 is branched from ultrapure water supply pipe 10 for supplying ultrapure water to the point of use. On-off valve 12 is provided in flow pipe 11. Flow pipe 11 provided downstream from on-off valve 12 may be composed of PFA tube 13 for decompression. Flow rate adjusting valve 23 is provided in the bypass line flow that is branched from pipe 11. Flow rate adjusting valve 23 adjusts the flow rate of the drainage exiting to the bypass line. Flow meter 24 is provided in the bypass line. Further, flow indicator 25 of the ultrasonic type is provided downstream from the branch point between the bypass line and flow pipe 11. Filtration device 20 is removably attached to the distal end of flow pipe 11 via pipe connector 21. As the ultrapure water in the ultrapure water supply pipe 10 is not contaminated at the time of attachment and removal of filtration device 20, it is preferable that filtration device 20 be attached to the pipe (such as flow pipe 11) branched from ultrapure water supply pipe 10. Filtration device 20 is, for example, a centrifugal filtration device.

Fine particle capturing membrane 22 is attached to the inside of filtration device 20. Inside filtration device 20, the ultrapure water that is the water to be analyzed is caused to flow from flow pipe 11 through pipe connector 21. Fine particle capturing membrane 22 captures fine particles in the ultrapure water that flows into filtration device 20 from flow pipe 11. Fine particle capturing membrane 22 is removably attached to filtration device 20. Differential pressure regulating valve 26 is provided downstream from filtration device 20. Differential pressure adjusting valve 26 is an on-off valve for airflow adjustment in filtration device 20. Further downstream, integrating flow meter 27 is provided for measuring the integrated flow rate of the water to be analyzed flowing through fine particle capturing membrane 22. Analysis target water that has flowed through fine particle capturing membrane 22 in filtration device 20 supplied via pipe connector 21 is drained to the outside as filtered water.

Once the water to be analyzed has passed through fine particle capturing membrane 22 over a predetermined period of time, at least one of quantitative analysis and qualitative analysis is performed on the fine particles captured by fine particle capturing membrane 22. Here, the quantitative accuracy of the fine particles in the water to be analyzed also depends on the integrated flow rate of water flow through fine particle capturing membrane 22. The amount of water that flows through fine particle capturing membrane 22 changes according to pressure fluctuation of the water to be analyzed. Therefore, it does not always match the actual integrated flow rate even if the flow rate is multiplied by the water flow time if the flow rate is adjusted at the start of water flow. Therefore, in filtration device 20 of the present embodiment, integrating flow meter 27 is provided downstream from filtration device 20 (fine particle capturing membrane 22) in order to calculate the actual integrated flow rate of the water to be analyzed that flows through fine particle capturing membrane 22. In this way, an accurate integrated flow rate value can be obtained. The reason for providing integrating flow meter 27 downstream from fine particle capturing membrane 22 with respect to the flow direction of the water to be analyzed is to avoid the influence of contamination from integrating flow meter 27. The flow rate to fine particle capturing membrane 22 is not adjusted based on the measured value of integrating flow meter 27.

After water to be analyzed has passed through filtration device 20 of the present embodiment over a predetermined period, the flow of water is stopped. Then, fine particle capturing membrane 22 is removed from filtration device 20. In order to stop the flow of water to filtration device 20, for example, flow rate adjusting valve 23 may be fully opened to cause the water to pass from PFA tube 13 only to the bypass line. As will be described later, quantification of fine particles captured in fine particle capturing membrane 22 may be performed immediately after removal, or may be performed according to a later request after a certain amount of time has elapsed. It is preferable that fine particle capturing membrane 22 be sealed and stored until the time of removing and performing the quantification so as not to contaminate fine particle capturing membrane 22 or to allow the fine particles to flow out of fine particle capturing membrane 22.

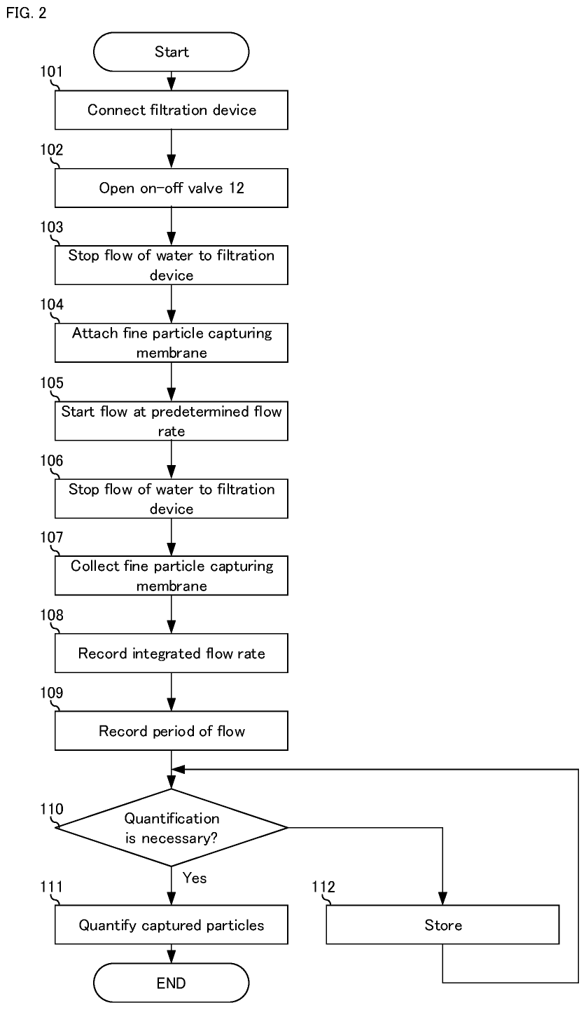

Next, a water quality management method using filtration device 20 shown in FIG. 1 will be described with reference to FIG. 2. Here, a case is described in which water quality control is performed by performing quantitative analysis of fine particles contained as impurities in, as the water to be analyzed, the ultrapure water that flows through ultrapure water supply pipe 10. Here, as an analysis for performing water quality control, a qualitative analysis may be performed, or a quantitative analysis and a qualitative analysis may be performed. First, in Step 101, filtration device 20 is connected to flow pipe 11 via pipe connector 21. At this time, fine particle capturing membrane 22 is not attached to filtration device 20. After filtration device 20 is connected to flow pipe 11, on-off valve 12 is opened and filtration device 20 is flushed (blown out) in Step 102. After flushing is performed for a period of time, the flow of water to filtration device 20 is stopped in Step 103. To stop the flow of water to filtration device 20, for example, flow rate adjusting valve 23 may be fully opened as described above. Subsequently, a pre-washed fine particle capturing membrane 22 is attached to filtration device 20 in Step 104. Then, in Step 105, the flow of water to be analyzed to filtration device 20 (fine particle capturing membrane 22) is started by adjusting the degree of opening of flow rate adjusting valve 23 in the closing direction. At this time, the degree of opening of flow rate adjusting valve 23 is adjusted based on the value displayed by flow display 25. Thus, the flow rate of the water to be analyzed flowing through filtration device 20 (fine particle capturing membrane 22) is adjusted. Then, when water to be analyzed has passed through fine particle capturing membrane 22 over a predetermined period of time, the flow of water to filtration device 20 is stopped in Step 106. The specific method of stopping the flow of water to filtration device 20 is as described above. Thereafter, in Step 107, fine particle capturing membrane 22 is collected from filtration device 20. Fine particle capturing membrane 22 in which fine particles have been captured by passing water to be analyzed is also referred to as a fine particle capturing membrane sample. FIG. 2 is a flow chart focusing on a particular filtration device 20. At the time that fine particle capturing membrane 22 is collected in Step 107, fine particle capturing membrane 22 for replacement is attached to filtration device 20, and the flow of water to filtration device 20 to which fine particle capturing membrane 22 for replacement is attached is resumed. By doing so, the management of water quality can be carried out over a continuous period of time. Incidentally, on-off valve 12 is usually kept in an open state. Flushing is performed even when fine particle capturing membrane 22 is not attached to filtration device 20. Further, on-off valve 12 is closed when changing the sampling point such as when removing PFA tube 13 from flow pipe 11. Incidentally, it is also possible to install a member for preventing the washing water from stagnating in filtration device 20 when washing the portion downstream from the branch point of the bypass line of flow pipe 11.

When fine particle capturing membrane 22 is collected, the value of the integrated flow rate measured by integrating flow meter 27 is recorded in Step 108. Further, in Step 109, the time period during which water passed through filtration device 20 (e.g., from what time on which month and date to what time on which month and date) is recorded. The value and period of the integrated flow rate may be filled in or recorded on, for example, a physical tag (e.g., a handwritten label, printed label, or IC (integrated circuit) chip) and attached to fine particle capturing membrane 22. In a case in which a serial number or the like is given to the fine particle capturing membrane 22, for example, the recording of the value and the period of the integrated flow rate may be managed by storing the serial number and the integrated flow rate in association with the water flow period in the database. Incidentally, when measuring the integrated flow manually without using integrating flow meter 27, the order of the process of Step 107 and the process of Step 108 is switched. It is then determined in Step 110 whether it is necessary to perform the quantification of the fine particles at that time. If a routine analysis is being performed, quantification is necessary and the process continues to Step 111. If there is no need for quantification at that time but there is a possibility that quantification will be performed later for a failure analysis, fine particle capturing membrane 22 is stored in Step 112 and the process returns to Step 110. Although a case in which fine particle capturing membrane 22 is stored in Step 112 has been described, filtration device 20 may also be stored. In that case, management is realized by attaching a physical tag to filtration device 20 as described above.

Performing the quantification of the captured particles in Step 111 completes the series of processes for a certain fine particle capturing membrane 22.

Quantification of captured particles in Step 111 may be performed by quantitative analysis or qualitative analysis using commonly known methods. Quantification of captured particles in Step 111 may be performed, for example, by observing and counting the captured particles using a scanning electron microscope (SEM), calculating the fine particles for the entire capturing membrane from the counted value, and calculating the particle concentration in the sample water to be measured based on the integrated flow meter of water that passed through the capturing membrane (volume). Further, the quantification of the captured particles in Step 111 may be performed by seeking the composition of a predetermined number of fine particles included in the range observed, or seeking the particle diameter and its particle size distribution of the fine particles.

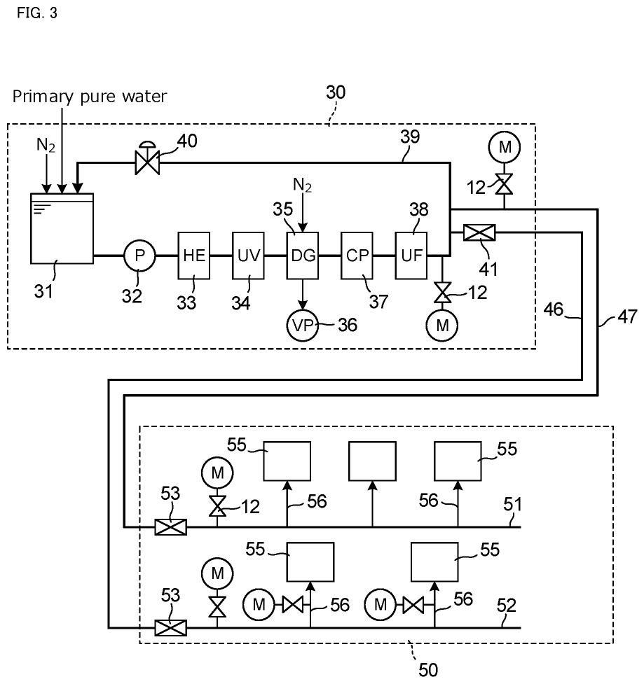

When a defect occurs in a product manufactured using ultrapure water, it may be suspected that the cause of the defect is the quality of the ultrapure water. For example, in the manufacturing of semiconductor devices, if a defect of a wafer is detected as a result of an inspection of the wafer that is performed several steps after using ultrapure water to wash the wafer in the semiconductor cleaning step, fine particles contained in the ultrapure water at the time of wafer cleaning may be suspected as a cause of the defect. In other words, when a problem occurs in a product, it is determined that a quantitative analysis of a fine particle capturing membrane sample is necessary for the period of the passage of a water that corresponds to the time when the product used water, and a quantitative analysis is performed. When an event suspected to be caused by such ultrapure water occurs, it is determined in Step 110 that quantification is necessary for, of fine particle capturing membranes 22 stored in Step 112, fine particle capturing membrane 22 in which the water flow period is at least the period corresponding to the event. Then, with respect to the fine particle capturing membrane sample of fine particle capturing membrane 22, the quantification of the captured particles is performed in Step 111. As a result, it is possible to determine whether the cause of the event such as a defect is fine particles in ultrapure water in the applicable period. Further, from the capturing membrane information (described later) of the fine particle capturing membrane sample subjected to quantification, it is possible to specify the site of the cause of an event such as a defect generated in a product or the like. For example, as shown in FIG. 3 (details will be described later), filtration devices 20 are provided at the exit of ultrafiltration device 38 of the ultrapure water producing apparatus 30, at the connection position between ultrapure water producing apparatus 30 and supply pipe 47, on main pipes 51 and 52 of manufacturing building 50, and on branch pipes 56 or the like that connect from main pipes 51 and 52 to ultrapure water use apparatuses 55. In this way, it is possible to identify from the quantitative results and the capturing membrane information which device and which member was the cause of an event such as a defect that occurred in a product, etc. Further, for example, a plurality of filtration devices 20 are provided at predetermined intervals on a long pipe such as supply pipe 46 or supply pipe 47 of FIG. 3. In this way, similarly, it is also possible to identify which point of supply pipe 46 or supply pipe 47 is the cause of an event such as a defect occurring in a product or the like. Filtration device 20 that takes a period of time that corresponds to an event as a water flow period is filtration device 20 having a water flow period that includes a period in which it came into contact with water to be analyzed at any time in the past when an event occurred in the manufacturing process of a product. In addition, the water flow period here is information indicating a time that can identify the date and time of water flow (the same applies in the following description). For example, the water flow period is information including at least one of a date and time at which water flow is started to filtration device 20 (fine particle capturing membrane 22) and a date and time at which water flow is terminated.

In this embodiment, since the water flow period is recorded for each of fine particle capturing membranes 22, even when the occurrence of a defect is found later, it is possible to easily find and analyze fine particle capturing membrane 22 of the water flow period corresponding to the defect from among fine particle capturing membranes 22 that are stored. In order to perform the defect analysis more precisely, it is preferable not only to perform the quantification of fine particle capturing membrane 22 of the water flow period corresponding to the period in which the defect occurred but also to perform the quantification of fine particle capturing membranes 22 of the water flow period corresponding to the period before and after the period in which the defect occurred.

According to this embodiment, it is possible to manage fine particles in ultrapure water as a continuous quantitative value for each predetermined period of time. Further, when there is a reduction of yield of the product or the like, it is possible to quickly determine whether the cause of reduction of yield was due to ultrapure water by comparing the production process history of the product with the water flow periods to the fine particle capturing membrane and the quantitative results of the particulate.

Next, an example of applying the water quality control method described above to a semiconductor device manufacturing plant will be described. FIG. 3 is a flow sheet showing the portion of the production and consumption of ultrapure water in a semiconductor device manufacturing plant. FIG. 3 also shows an example of the connection locations of filtration device 20 in a semiconductor device manufacturing plant.

In the semiconductor device manufacturing plant shown, ultrapure water producing apparatus (secondary system pure water producing apparatus) 30 that is supplied with primary pure water and that produces ultrapure water primary pure water, i.e. a subsystem, and manufacturing building 50 where the ultrapure water is actually used are provided separately. Ultrapure water producing apparatus 30 includes tank 31, pump (P) 32, heat exchanger (HE) 33, ultraviolet oxidation device (UV) 34, membrane degasser (DG) 35, non-regenerative ion exchanger (CP) 37, and ultrafiltration device (UF) 38. Tank 31 receives and temporarily stores primary pure water. Pump (P) 32 is provided at the outlet of tank 31. Heat exchanger (HE) 33 is provided at the outlet of pump 32. Ultraviolet oxidation device (UV) 34, membrane degasser (DG) 35, non-regenerative ion exchange device (CP) 37, and ultrafiltration device (UF) 38 each perform steps for producing ultrapure water. Ultraviolet oxidation device 34, membrane degasser 35, non-regenerative ion exchanger 37, and ultrafiltration device 38 are connected in a series to the outlet of heat exchanger 33 in that order. Vacuum pump (VP) 36 is connected to membrane degasser 35. The outlet water of ultrafiltration device 38 is ultrapure water, and a portion of the ultrapure water is supplied to manufacturing building 50 via supply pipes 46 and 47. The remaining ultrapure water not supplied to manufacturing building 50 is returned to tank 31 via circulation pipe 39. Valve 40 is provided on circulation pipe 39 in order to, for example, keep the water pressure constant along the path in which the ultrapure water circulates. Nitrogen (N₂) gas is supplied to tank 31 to purge oxygen and thus minimize dissolved oxygen in the ultrapure water. Since a nitrogen sweep is performed together with the removal of oxygen, nitrogen gas is also supplied to membrane degasser 35. The configuration and the arrangement of ultrapure water producing apparatus 30 is not limited to what is shown in the figure.

Ion adsorber 41 for capturing ultratrace amounts of ionic impurities and a fine particle removal filter (not shown) for capturing fine particles in the ultrapure water are provided on the ultrapure water producing apparatus 30 side of supply pipe 46 of supply pipes 46 and 47 to manufacturing building 50. Fine particle removal filter is provided further downstream from supply pipes 46 and 47 than ion adsorber 41. This ion adsorber 41 need not be provided.

In manufacturing building 50, main pipes 51 and 52 are provided for connecting to supply pipes 46 and 47, respectively. A plurality of ultrapure water using apparatuses 55 are connected to main pipes 51 and 52 via branch pipes 56. Ultrapure water using apparatuses 55 are, for example, a cleaning apparatus, an etching apparatus, an exposure apparatus, or the like. On the inlet sides of main pipes 51 and 52, ion adsorber 53 and the fine particle removal filters (not shown) are provided for capturing ultratrace amounts of ionic impurities and fine particles contained in the ultrapure water supplied from supply pipes 46 and 47, respectively. The fine particle removal filters are provided further downstream from supply pipes 46 and 47 than ion adsorber 53. Ion adsorber 53 may not necessarily be provided.

Examples of locations where filtration device 20 shown in FIG. 1 can be provided are shown by reference numeral M in FIG. 3. That is, in ultrapure water producing apparatus 30, filtration device 20 may be provided at the outlet of ultrafiltration device 38 or may be provided at the connection position with supply pipe 47. In manufacturing building 50, filtration device 20 may be provided in each of main pipes 51 and 52, or may be provided on branch pipes 56 that connect to respective ultrapure water using apparatuses 55. The locations and number of installations of filtration device 20 are not limited to those shown, and it is possible to install filtration device 20 in any location. Each filtration device 20 is connected to a pipe such that ultrapure water flows through on-off valve 12 in the same manner as shown in FIG. 1. On-off valve 12 is usually opened and closed when changing the sampling point. Drained water from filtration device 20 is preferably returned to the recovered water system if a recovered water system is provided in the semiconductor device factory.

Hereinafter, a method of utilizing the above-described filtration device will be described by way of example.

### First Example of a System

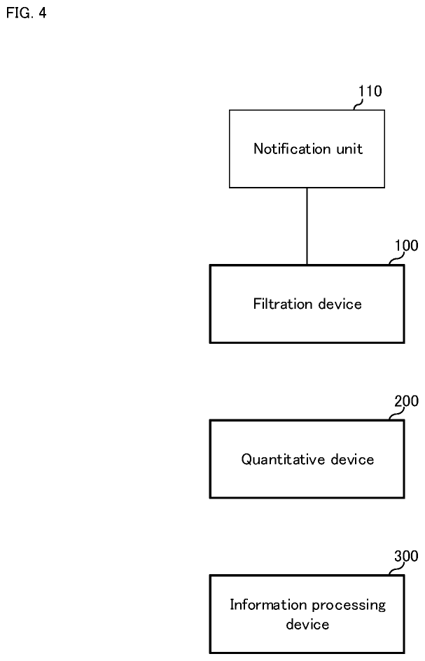

FIG. 4 is a diagram showing a first example of an information processing system using filtration device 20 shown in FIG. 1. Here, a case will be described in which quantitative analysis of fine particles is performed using a quantitative device as an analyzer. To perform the analysis of fine particles, a qualitative analysis may be performed using a qualitative device, or a quantitative analysis and a qualitative analysis may be performed using a quantitative device and a qualitative device.

The information processing system shown in FIG. 4 includes filtration device 100, quantitative device (analyzer) 200, and information processing device 300. Filtration device 100 corresponds to filtration device 20 shown in FIG. 1. Further, filtration device 100 is connected to notification unit 110. Notification unit 110 issues a predetermined notification such as, for example, a notification indicating that a predetermined period has elapsed after the particle capturing membrane (fine particle capturing membrane 22 shown in FIG. 1; hereinafter, the same) was attached to filtration device 100. Alternatively, after the fine particle capturing membrane is attached to filtration device 100, notification unit 110 issues a predetermined notification, such as, for example, a notification indicating that the integrated value measured by the integrating flow meter (integrating flow meter 27 shown in FIG. 1; hereinafter, the same) provided downstream from filtration device 100 has reached a predetermined value. Incidentally, filtration device 100 also includes integrating flow meter 27 shown in FIG. 1. At this time, notification unit 110 issues a notification prompting the removal of the fine particle capturing membrane from filtration device 100. Notification unit 110 may be provided inside filtration device 100. Notification unit 110 may display notification on a device such as another terminal device having an information display function.

Quantitative device 200 performs quantitative analysis of a fine particle capturing membrane in which fine particles are captured. The specific methods of quantitative analysis are as described above. A method of identifying a fine particle capturing membrane to be subjected to quantitative analysis will be described later.

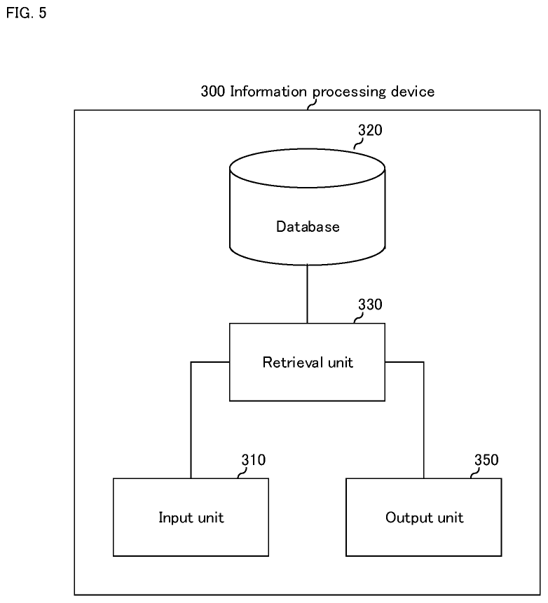

FIG. 5 is a diagram showing an example of the internal configuration of information processing device 300 shown in FIG. 4. As shown in FIG. 5, information processing device 300 shown in FIG. 4 includes input unit 310, database 320, retrieval unit 330, and output unit 350. Incidentally, FIG. 5 shows, of the components provided by information processing device 300 shown in FIG. 4, only the main components relating to the present embodiment.

Input unit 310 supplies the input information to information processing device 300 based on an operation accepted from the outside. Specifically, input unit 310 receives a predetermined operation from the outside, and receives information based on the received operation. The information input unit 310 receives is, for example, information for instructing the retrieval of the fine particle capturing membrane sample when defects of a wafer are detected in the semiconductor device manufacturing process and it is determined that it is necessary to perform a quantitative analysis of the fine particle capturing membrane sample of the water flow period corresponding to the time of use of the wafer cleaning water. Input unit 310 may be, for example, a keyboard, a mouse, a touch panel, or the like. Input unit 310 may display a GUI (Graphical User Interface) prompting input of predetermined information, and input information based on an operation performed according to the display. Further, the information recorded by filtration device 100 or the information reported by notification unit 110 is transmitted to information processing device 300, and input unit 310 may receive and then supply the information that has been transmitted.

Database 320 stores the period information and the capturing membrane identification information in association with each other as the capturing membrane information. The period information indicates the period during which the fine particle capturing membrane was attached to the flow pipe (the period during which water flowed into fine particle capturing membrane). Further, the period information includes information such as the date and time when the water started to flow into the fine particle capturing membrane and the date and time when the water flow was terminated. The capturing membrane identification information is information uniquely conferred to the fine particle capturing membrane. In addition, database 320 stores the installation information in association with the capturing membrane information. The installation information is information of filtration device 100 to which a fine particle capturing membrane was attached. The method of registering the information to database 320 is not particularly limited. For example, when registering the period information, information including the date and time at the times of opening and closing on-off valve 12 may be transmitted to database 320 and then stored (registered) as period information. Further, when registering the period information, information including the date and time at the time when the water to be analyzed starts to flow and the time when the flow ends to integrating flow meter 27 may be transmitted to database 320 and then stored (registered) as period information. In addition, when the capturing membrane identification information is registered, a tab for identification such as a bar code or a two-dimensional code may be attached to fine particle capturing membrane 22, and the attached identification tab may then be read by a code reader (reading device), and the read information then transmitted to database 320 and stored (registered) as the capturing membrane information.

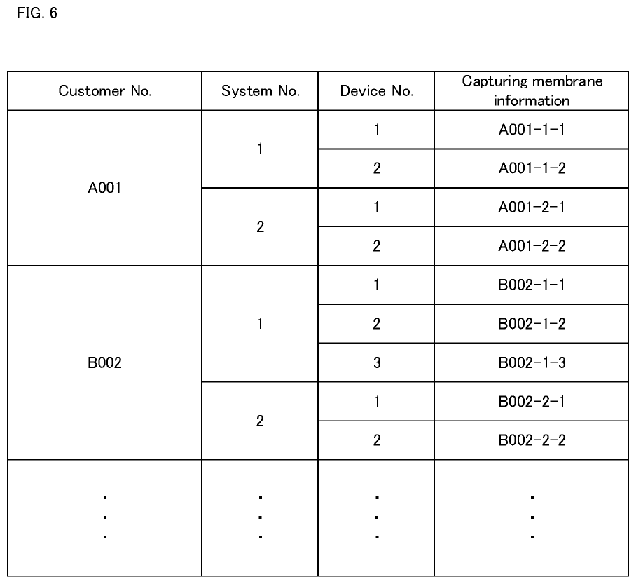

FIG. 6 is a diagram showing an example of the association between the installation information and the capturing membrane information stored in database 320 shown in FIG. 5. As shown in FIG. 6, in database 320 shown in FIG. 5, “Customer No.,” “System No.,” “Device No.,” and “Capturing Membrane Information” that can identify the installation location of filtration device 100 to which the fine particle capturing membrane is attached are stored in association with each other. “Customer No.,” “System No.,” and “Device No.” are used together as installation information. “Customer No.” is customer identification information uniquely conferred to a customer for whom filtration device 100 is installed to which a fine particle capturing membrane is attached. “System No.” is system identification information uniquely conferred to a system constructed in a customer's facility. “Device No.” indicates an apparatus in which system filtration device 100 is installed and is device identification information uniquely assigned to the installed device. Thus, using “Customer No.,” “Customer No.,” and “Device No.” enables identification of the installation location of filtration device 100 to which a fine particle capturing membrane is attached. Details of the “capturing membrane information” will be described later.

For example, as shown in FIG. 6, customer No. “A001,” system No. “1,” device No. “1,” and capturing membrane information “A001-1-1” are stored in association with each other. This indicates that the fine particle capturing membrane indicated by the capturing membrane information “A001-1-1” is attached (was attached) to the device that was given the device identification information “1” that is installed in the system given the system identification information “1” that is constructed in the facility of the customer given the customer identification information “A001.” Further, customer No. “A001,” system No. “1,” device No. “2,” and capturing membrane information “A001-1-2” are stored in association with each other. This indicates that the fine particle capturing membrane indicated by the capturing membrane information “A001-1-2” is attached (was attached) to the device given device identification information “2” that is installed in the system given the system identification information “1” that is constructed in the facility of the customer given the customer identification information “A001.” Further, customer No. “A001,” system No. “2,” device No. “1,” and capturing membrane information “A001-2-1” are stored in association with each other. This indicates that the fine particle capturing membrane indicated by capturing membrane information “A001-2-1” is attached (was attached) to the device given device identification information “1” that is installed in the system given system identification information “2” that is constructed in the facility of the customer given customer identification information “A001.” Further, customer No. “A001,” system No. “2,” device No. “2,” and capturing membrane information “A001-2-2” are stored in association with each other. This indicates that the fine particle capturing membrane indicated by capturing membrane information “A001-2-2” is attached (was attached) to the device given device identification information “2” that is installed in the system given system identification information “2” that is constructed in the facility of the customer given customer identification information “A001.”

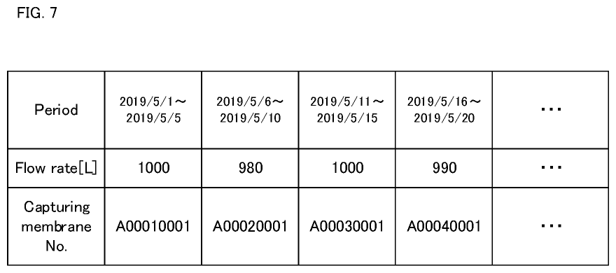

FIG. 7 is a diagram showing an example of the association between the period information and the capturing membrane identification information stored in database 320 shown in FIG. 5. This association is the capturing membrane information described above. The capturing membrane information shown in FIG. 7 is one item of the capturing membrane information shown in FIG. 6 (capturing membrane information “A001-1-1”). As shown in FIG. 6, when nine items of capturing membrane information are stored in database 320, nine items of capturing membrane information associated as shown in FIG. 7 are stored in database 320. Thus, for example, the capturing membrane information shown in FIG. 7 corresponds to one item “A001-1-1” of the capturing membrane information shown in FIG. 6.

As shown in FIG. 7, in database 320 shown in FIG. 5, “Period,” “Flow rate [L],” and “Capturing membrane No.” are placed in association and are stored as one item of capturing membrane information. “Period” is period information indicating a period of time during which a fine particle capturing membrane was attached to filtration device 100. “Flow rate [L]” is the integrated amount of water flow in that period. “Capturing membrane No.” is the capturing membrane identification information uniquely conferred to the fine particle capturing membrane. Incidentally, the flow rate is the integrated value measured by the integrating flow meter in that period.

For example, as shown in FIG. 7, the period “2019/5/1 to 2019/5/5,” the flow rate “1000 [L],” and the capturing membrane No. “A00010001” are stored in association with each other. This indicates that the fine particle capturing membrane to which the capturing membrane identification information “A00010001” was given was attached to filtration device 100 for five days from May 1, 2019 to May 5, 2019, during which time the amount of water flow to be analyzed that flowed through the fine particle capturing membrane was 1000 [L]. In addition, the period “2019/5/6 to 2019/5/10,” the flow rate “980 [L],” and the capturing membrane No. “A00020001” are stored in association with each other. This indicates that the fine particle capturing membrane to which the capturing membrane identification information “A00020001” was given was attached to filtration device 100 for five days from May 6, 2019 to May 10, 2019, during which time the amount of water flow to be analyzed that flowed through the fine particle capturing membrane was 980 [L]. In addition, the period “2019/5/11 to 2019/5/15,” the flow rate “1000 [L],” and the capturing membrane No. “A00030001” are stored in association with each other. This indicates that the fine particle capturing membrane to which the capturing membrane identification information “A00030001” was given was attached to filtration device 100 for five days from May 11, 2019 to May 15, 2019, during which time the amount of water flow to be analyzed that flowed through the fine particle capturing membrane was 1000 [L]. In addition, the period “2019/5/16 to 2019/5/20,” the flow rate “990 [L],” and the capturing membrane No. “A00040001” are stored in association with each other. This indicates that the fine particle capturing membrane to which the capturing membrane identification information “A00040001” was given was attached to filtration device 100 for five days from May 16, 2019 to May 20, 2019, during which time the amount of water flow to be analyzed that flowed through the fine particle capturing membrane was 990 [L]. These associations were registered and stored after the respective fine particle capturing membranes were removed from filtration device 100. The registration method may be one in which this information is transmitted from filtration device 100 to information processing device 300 and registered. Further, this registration method may be one in which this information is registered via another medium. Incidentally, in the example shown in FIG. 5, “period” is period information that shows only the information indicating the date, but information indicating the date and time that includes the time (hour) is also included. In other words, the period information includes information indicating the date and time at which the fine particle capturing membrane was attached to filtration device 100 and information indicating the date and time at which the fine particle capturing membrane was removed from filtration device 100

Retrieval unit 330 retrieves the capturing membrane identification information from database 320 based on the date and time information (information related to the time when a problem occurred in a product that used water) included in the input information received by input unit 310. Specifically, retrieval unit 330 retrieves the period that includes the date and time that was indicated by the date and time information included in the input information received by input unit 310 from database 320 and retrieves from database 320 the capturing membrane identification information associated with the retrieved period. At this time, retrieval unit 330 retrieves the capturing membrane information from database 320 based on the installation information of the filtration device included in the input information received by input unit 310 and retrieves the capturing membrane identification information from database 320 based on the retrieved capturing membrane information and the date and time information. For example, if the customer No. is “A001,” the system No. is “1,” and the date and time information is “May 3, 2019” for installation information included in the input information, retrieval unit 330 retrieves the capturing membrane information that has customer No. “A001,” system No. “1,” and system No. “1” from database 320, and retrieves the capturing membrane No. “A00010001” that corresponds to “May 1, 2019-May 5, 2019” for the period including the date and time information “May 3, 2019” from the association of the retrieved capturing membrane information “A001-1-1.”

The configuration of the system in the customer's facility may be registered in database 320 in advance, and retrieval unit 330 may perform retrieval based on the configuration of the system. For example, if it is believed that the device of customer No. “A001,” system No. “1,” and device No. “1” and the device of customer No. “A001,” system No. “1,” and device No. “2” may affect each other based on the configuration of the system, retrieval unit 330 may also retrieve the capturing membrane information regarding the device of customer No. “A001,” system No. “1,” and device No. “2” even when the installation information included in the input information is only customer No. “A001,” system No. “1,” and device No. “1.” Here, in order to determine whether or not there is mutual influence, a determination model may be generated using machine learning based on the configuration of the system and past determination results, and the determination may then be performed using the determination model. For example, it may be determined that devices affect each other when the device of customer No. “A001,” the system No. “1” and the device No. “1”, and the device of the customer No. “A001,” system No. “1,” and device No. “2” are installed side by side in a series, or when a relationship is recognized between the analytical results of the devices based on previous analytical results, etc. Thus, when the cause of a product defect is the pollutants contained in the ultrapure water, performing an analysis on devices that affect each other can determine if a device among the plurality of devices provided in a system is generating pollutants, i.e., can identify which of the devices is generating the pollutants.

Output unit 350 supplies the capturing membrane identification information retrieved by retrieval unit 330. The method of supplying the capturing membrane identification information performed by output unit 350 may be, for example, transmission to another device, screen display, audio output, or printing.

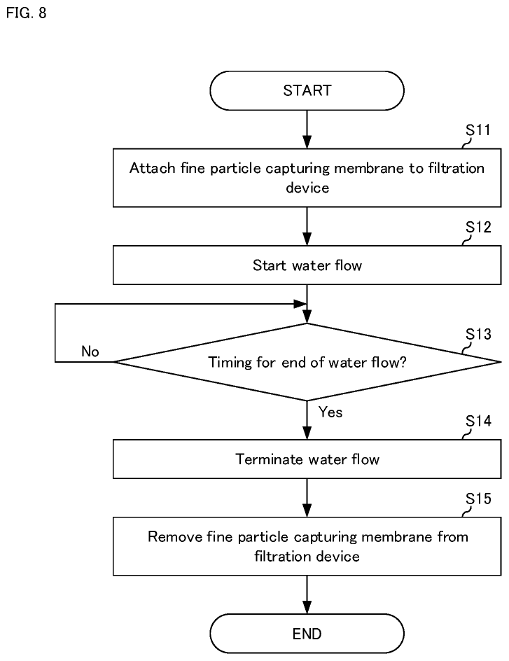

The information processing method in the information processing system shown in FIG. 4 will be described below. FIG. 8 is a flowchart for explaining an example of processing in filtration device 100 among the information processing methods in the information processing system shown in FIG. 4.

First, a fine particle capturing membrane is attached to filtration device 100 (Step S11). Subsequently, the water flow to flow pipe 11 is started (Step S12). After the fine particle capturing membrane has been attached to filtration device 100, water flow to the fine particle capturing membrane is started by adjusting the opening degree of flow rate adjusting valve 23 shown in FIG. 1 in the closing direction from the fully opened state.

Thereafter, it is determined whether the timing of the end of the water flow has arrived (Step S13). Here, it is determined that the timing of the water flow has arrived when a predetermined period has elapsed from the start of the water flow or when the integrated value of the amount of water flow has reached a predetermined value. The passage of a predetermined period may be determined using a timer. The measurement of the integrated value of the water flow amount may be performed using an integrating flow meter. When these timings have been reached, notification unit 110 may report that the timing has been detected to the manager, the operator, or the maintenance person (hereinafter, referred to as “the manager” or the like) of the system on a display or the like. Thereafter, water flow to the fine particle capturing membrane is terminated (Step 14). At this time, flow rate adjusting valve 23 shown in FIG. 1 is set to the fully opened state. Alternatively, the person who has received the notification adjusts the opening of flow rate adjusting valve 23. The fine particle capturing membrane is then removed from filtration device 100 (Step S15). At this time, a new fine particle capturing membrane is attached to filtration device 100. In addition, the timer and the integrating flow meter are reset every time a fine particle capturing membrane is attached to filtration device 100 (a fine particle capturing membrane is replaced). Incidentally, the time from the timing of stopping the flow of water to filtration device 100 for the exchange of the fine particle capturing membrane to the timing of the start of the flow of water is to be as short as possible so that the continuity of the flow period with respect to the fine particle capturing membrane can be ensured.

Information such as the water flow period of the removed fine particle capturing membrane is stored in database 320 of information processing device 300. The information to be stored is the information shown in FIG. 7, and each of the plurality of items of information for each of the fine particle capturing membranes is stored in association with each other. This storage is performed through input unit 310 of information processing device 300. Further, the removed fine particle capturing membrane is given capturing membrane identification information and stored in a predetermined storage place.

If a quantitative analysis is subsequently required, a search request is made to information processing device 300. When a defect occurs in a product or the like produced using ultrapure water that is the object of analysis, quantitative analysis must be performed to confirm whether the cause of the defect is in the water quality of the ultrapure water. To do so, it is necessary to retrieve and remove the target fine particle capturing membrane sample (that is, the fine particle capturing membrane sample through which water passed during the water flow period that corresponds to the time when the product in which a problem occurred used water).

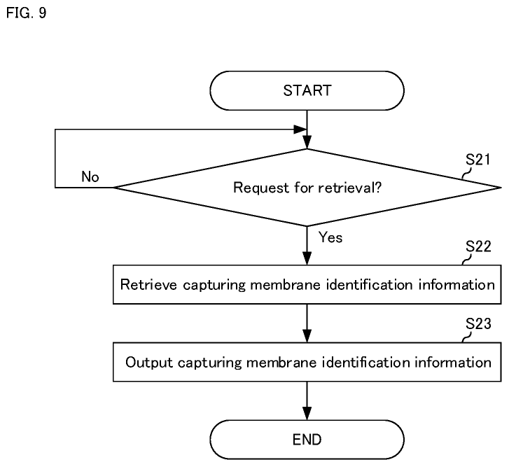

FIG. 9 is a flowchart for explaining an example of the search processing in information processing apparatus 300 among the information processing methods in the information processing system shown in FIG. 4.

Input unit 310 determines whether or not there is a request for retrieval of a fine particle capturing membrane (Step S21). This request may be based on a predetermined operation performed by the manager of the system upon input unit 310 for requesting the retrieval of a fine particle capturing membrane and is received by input unit 310. This predetermined operation includes the installation information and the date and time information of the target device (the device in which a failure occurred). Input unit 310 supplies, of the received information, the installation information and the date and time information to retrieval unit 330. Retrieval unit 330 retrieves the capturing membrane identification information from database 320 based on the installation information and the date and time information supplied from input unit 310 (Step S22). Specifically, for example, retrieval unit 330 retrieves the capturing membrane information from database 320 based on the installation information supplied from input unit 310. Retrieval unit 330 then retrieves from database 320, of the retrieved capturing membrane information, the capturing membrane identification information associated with a period that includes the date and time information supplied from input unit 310. Output unit 350 then supplies the capturing membrane identification information retrieved by retrieval unit 330 (Step S23).

Thereafter, the operator or the like secures the fine particle capturing membrane to which the capturing membrane identification information supplied from output unit 350 is conferred from the storage location, and quantification is performed using quantitative device 200. The concentration of fine particles in the water to be analyzed is then calculated using the result of the quantitative analysis and the integrated value measured by the integrating flow meter. The result of the quantification and the concentration of fine particles in the water to be analyzed are provided from the operator or the like to the desired donor.

In this way, in a system for performing water quality management, filtration devices provided with a fine particle capturing membranes is removed at predetermined timings. The fine particle capturing membranes that were provided in the removed filtration devices are stored, and a fine particle capturing membrane that was attached to a filtration device of a designated installation location and period is retrieved from among the stored fine particle capturing membranes. Quantitative analysis of the retrieved fine particle capturing membrane is performed to provide results. It is thus possible to perceive the treatment status of the water to be analyzed at specified locations and specified dates and times.

### Second System Example

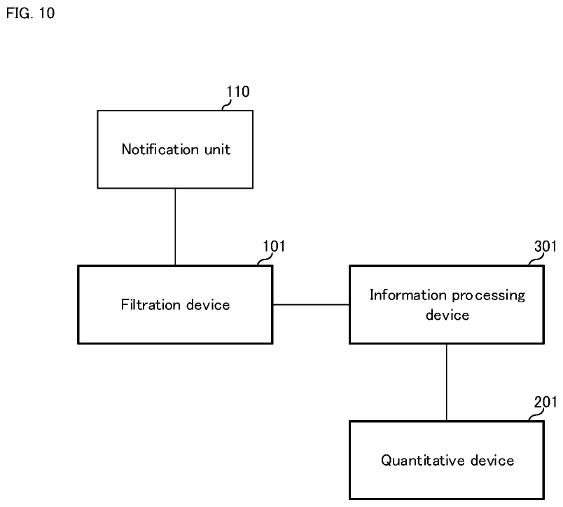

FIG. 10 is a diagram showing a second example of an information processing system utilizing filtration device 20 shown in FIG. 1. Here, a case in which quantitative analysis of fine particles is performed using a quantitative device as an analyzer will be described. To perform the analysis of fine particles, a qualitative analysis may be performed using a qualitative device, or a quantitative analysis and a qualitative analysis may be performed using a quantitative device and a qualitative device.

The information processing system shown in FIG. 10 includes filtration device 101, quantitative device (analyzer) 201, and information processing device 301. Filtration device 101 corresponds to filtration device 20 shown in FIG. 1. Further, filtration device 101 transmits the date and time information of the attachment of a fine particle capturing membrane to a flow pipe, the date and time information of the detachment from the flow pipe, and the identification information of the fine particle capturing membrane to information processing device 301. Further, filtration device 101 is connected to notification unit 110. Notification unit 110 issues a predetermined notification such as, for example, a notification indicating that a predetermined period has elapsed after the fine particle capturing membrane provided in filtration device 100 was attached to the flow pipe. Alternatively, after the fine particle capturing membrane provided in filtration device 101 is attached to the flow pipe, when the integrated value measured by the integrating flow meter provided in filtration device 101 becomes a predetermined value, notification unit 110 issues a predetermined notification such as a notification or the like indicating this fact. At this time, notification unit 110 issues a notification prompting the removal of the fine particle capturing membrane from the flow pipe. Notification unit 110 may be provided inside filtration device 101. Notification unit 110 may display on a device such as another terminal device having an information display function.

Quantitative device 201 performs quantitative analysis of fine particles captured by the fine particle capturing membrane. Specific methods of quantitative analysis are as described above. A method of identifying a fine particle capturing membrane to be subjected to quantitative analysis will be described later. Quantitative device 201 provides the result of performing quantitative analysis to information processing device 301. The method of providing the result may be a method in which quantitative device 201 transmits information indicating the analysis result to information processing device 301, or may be a method in which quantitative device 201 provides information via another medium.

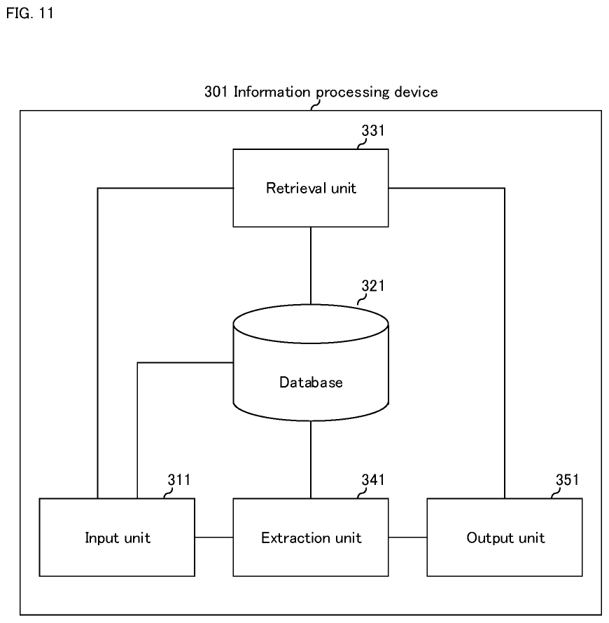

FIG. 11 is a diagram showing an example of an internal configuration of information processing device 301 shown in FIG. 10. Information processing device 301 shown in FIG. 10 includes input unit 311, database 321, retrieval unit 331, extraction unit 341, and output unit 351, as shown in FIG. 11. Incidentally, of the components provided for information processing device 301 shown in FIG. 10, FIG. 11 shows only the main components relating to the present embodiment.

Input unit 311 supplies input information to information processing device 301 based on an operation accepted from the outside. Specifically, input unit 311 receives a predetermined operation from the outside and supplies information based on the received operation. The information input unit 311 supplies is, for example, information for instructing the retrieval of a fine particle capturing membrane sample when a defect of a wafer have been detected in a semiconductor device manufacturing process and it has been determined that a quantitative analysis must be performed of the fine particle capturing membrane sample of the water flow period that corresponds to the time of using wafer cleaning water. Input unit 311 may be, for example, a keyboard, a mouse, a touch panel, or the like. Input unit 311 may display a GUI prompting input of prescribed information and may receive information based on an operation that is performed in accordance with the display. Further, information recorded by filtration device 101 or information reported by notification unit 110 is transmitted to information processing device 301, and input unit 311 may receive by receiving the information that has been transmitted.

Database 321 stores the period information in association with the capturing membrane identification information as the capturing membrane information. The period information indicates a water flow period (including the date and time at which water flow to the fine particle capturing membrane was started, the date and time at which water flow was terminated, and the like). The capturing membrane identification information is identification information uniquely conferred to a fine particle capturing membrane. Further, database 321 stores installation information in association with capturing membrane information. Installation information is information of filtration device 101 to which a fine particle capturing membrane has been attached. The state of storage of this information is the same as that shown in FIGS. 6 and 7. In addition, database 321 may store, for example, an analysis result transmitted from quantitative device 201 when a quantitative analysis has been performed by determining that a quantitative analysis of a fine particle capturing membrane sample for a period of water passage corresponds to a time when a problem occurred in a product that uses water. At this time, the analysis result transmitted from quantitative device 201 is stored in database 321 via input unit 311.

Retrieval unit 331 retrieves the capturing membrane identification information from database 321 based on the date and time information that is included in input information received by input unit 311. Specifically, retrieval unit 331 retrieves from database 321 the period that includes the date and time indicated by the date and time information included in the input information received by input unit 311 and retrieves from database 321 the capturing membrane identification information associated with the retrieved period. At this time, retrieval unit 331 retrieves the capturing membrane information from database 321 based on the installation information of the filtration device included in the input information received by input unit 311. Retrieval unit 331 further retrieves the capturing membrane identification information from database 321 based on the retrieved capturing membrane information and the date and time information. For example, if the customer No. is “A001,” the system No. is “1,” and the date and time information is “May 3, 2019” for installation information included in the input information, retrieval unit 331 retrieves the capturing membrane information that has customer No. “A001,” system No. “1,” and device No. “1” from database 321 and retrieves the capturing membrane No. “A00010001” that corresponds to the period “May 1, 2019-May 5, 2019” that includes the date and time information “May 3, 2019” based on the association with the retrieved capturing membrane information “A001-1-1”.

The configuration of the system in the customer's facility may be registered in database 321 in advance, and retrieval unit 331 may perform retrieval based on the configuration of the system. For example, if it is believed that the device of customer No. “A001,” system No. “1,” and device No. “1” and the device of customer No. “A001,” system No. “1,” and device No. “2” may affect each other based on the configuration of the system, retrieval unit 331 may also retrieve the capturing membrane information regarding the device of customer No. “A001,” system No. “1,” and device No. “2” even if the customer No. is “A001,” the system No. is “1” and the device No. is “1” of the installation information included in the input information. Here, in order to determine whether or not there is mutual influence, a determination model may be generated using machine learning based on the configuration of the system and past determination results, and the determination of influence may then be performed using the determination model. For example, when the device of customer No. “A001,” system No. “1,” and device No. “1,” and device of customer No. “A001,” system No. “1,” and device No. “2” are installed side by side in a series, or when a relationship is recognized between the analytical results of the two devices based on previous analytical results, etc., it may be determined that the two devices affect each other. Thus, analyses on devices that affect each other are performed. Therefore, when the cause of product defects is pollutants contained in ultrapure water, it is possible to determine if any of the plurality of devices provided in the system is generating pollutants, i.e., to identify the device that is generating the pollutants.

Extraction unit 341 extracts provided information, which is information corresponding to input information based on the result of the quantitative analysis that is provided (transmitted) from quantitative device 201 for a fine particle capturing membrane that is given capturing membrane identification information and retrieved by retrieval unit 331. Here, the input information may include, for example, specific analysis content. In that case, extraction unit 341 extracts from the results of quantitative analysis performed by quantitative device 201 the result corresponding to the analysis content contained in input information. When the quantitative analysis provided (transmitted) from quantitative device 201 is stored in database 321, extraction unit 341 extracts from the results of quantitative analysis stored in database 321 the provided information, which is information corresponding to the input information.

Output unit 351 supplies the capturing membrane identification information retrieved by retrieval unit 331. Output unit 351 supplies, as the provided information, the result of the quantitative analysis performed by quantitative device 201 on the fine particle capturing membrane to which the capturing membrane identification information retrieved by retrieval unit 331 was given. Further, when extraction unit 341 extracts the provided information, which is the information corresponding to the input information, from the results of quantitative analysis performed by quantitative device 201, output unit 351 supplies the provided information extracted by extraction unit 341. The method of supplying the provided information performed by output unit 351 may be, for example, transmission to another device, or may be screen display, audio output, printing, or lighting or blinking of a prescribed lamp.

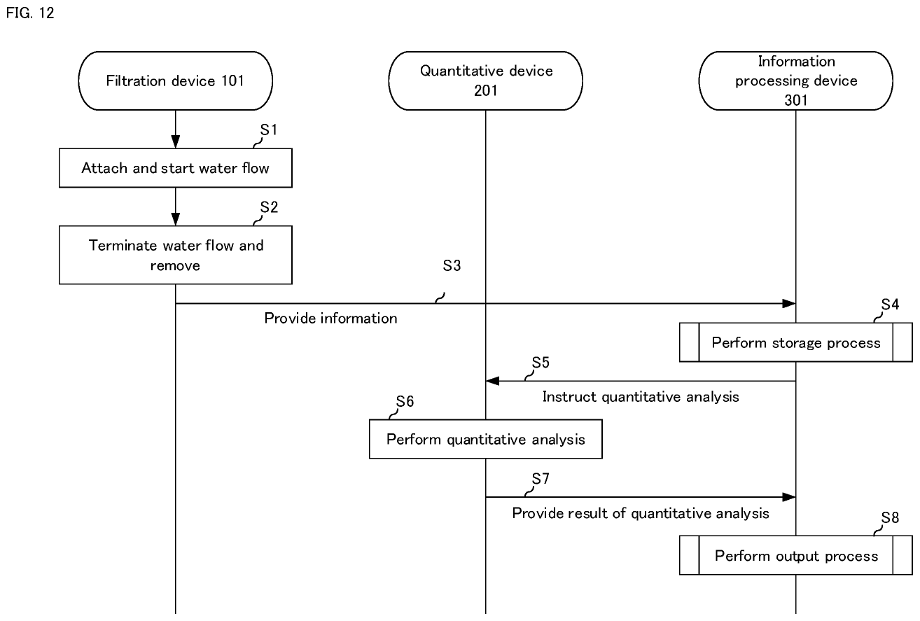

FIG. 12 is a sequence diagram for explaining an example of an information processing method in the information processing system shown in FIG. 10.

First, a fine particle capturing membrane is attached to filtration device 101, and water flow is started to filtration device 101 (Step S1). After the fine particle capturing membrane has been attached to filtration device 101, water flow to the fine particle capturing membrane is started by adjusting the opening degree of flow rate adjusting valve 23 shown in FIG. 1 in the closing direction from the fully open state. Water flow to filtration device 101 is subsequently terminated when a predetermined period has elapsed or when the integrated value measured by the integrating flow meter 27 shown in FIG. 1 reaches a predetermined value. The determination of the termination of water flow to the filtration device 101 is not performed by carrying out a process directly triggered by the passage of a prescribed time period or the detection that a predetermined value of the integrated value has been reached, but these detections activate a notification process to that effect, and the person receiving this notification adjusts the degree of opening of flow rate adjusting vale 23 to terminate the water flow to filtration device 101. At this time, the opening degree of flow rate adjusting valve 23 shown in FIG. 1 is fully opened. Here, filtration device 101 has a timer and measures the time from the start of water flow to the fine particle capturing membrane, and when a preset time has elapsed, notification unit 110 reports this fact and the water flow to filtration device 101 is terminated. Alternatively, when the integrated value measured by integrating flow meter reaches a preset value, notification unit 110 reports this fact, and the water flow to filtration device 101 is terminated. The report issued by notification unit 110 is directed to the manager or the like of the system, and such persons are supposed to terminate the water flow by setting the opening degree of flow rate adjusting valve 23 to the fully open state. The report issued by notification unit 110 may also be directed to flow rate adjusting valve 23, and flow rate adjusting valve 23 may automatically assume the fully open state to terminate water flow. The fine particle capturing membrane is then removed from filtration device 101 (Step S2). At this time, a new fine particle capturing membrane is attached to filtration device 101. In addition, the timer and the integrating flow meter are reset each time a fine particle capturing membrane is attached to filtration device 101 (each time the fine particle capturing membrane is replaced).

Thereafter, information of the removed fine particle capturing membrane is provided to information processing device 301 (Step S3). The information provided is the period information of the removed fine particle capturing membrane, the integrated value measured by the integrating flow meter, the capturing membrane identification information of the fine particle capturing membrane and the installation information of filtration device 101 to which the fine particle capturing membrane was attached. The method of providing this information may be a method in which filtration device 101 transmits and provides this information to information processing device 301, or may be a method in which filtration device 101 provides the information via another medium. The timing at which the information of the fine particle capturing membrane is provided to information processing device 301 may be after Step S1. The information provided in this case is information indicating the date and time when the fine particle capturing membrane was attached to filtration device 101 and water flow was started to filtration device 101. The storage process is then performed in information processing device 301 (Step S4). Note that the removed fine particle capturing membrane is stored at a predetermined place so as to enable identification using the capturing membrane identification information.

Subsequently, when information processing device 301 instructs quantitative device 201 to perform quantitative analysis (Step S5), quantitative device 201 performs the quantitative analysis (Step S6). At this time, information processing device 301 specifies the capturing membrane identification information to instruct quantitative device 201 to perform the quantitative analysis, and quantitative device 201 performs the quantitative analysis of the fine particles captured by the fine particle capturing membrane to which the indicated capturing membrane identification information was conferred. This method of instructing quantitative analysis may be one in which information processing device 301 instructs by transmitting to quantitative device 201 information indicating that quantitative analysis is required, or may be one in which information is provided via another medium. When the quantitative analysis is completed, quantitative device 201 provides the result to information processing device 301 (Step S7). The method of providing the result of this quantitative analysis may be one in which quantitative device 201 sends and provides information indicating the result of the quantitative analysis to information processing device 300, or may be one in which the quantitative analysis is provided via another medium. Information processing device 301 then performs output processing (Step S8).

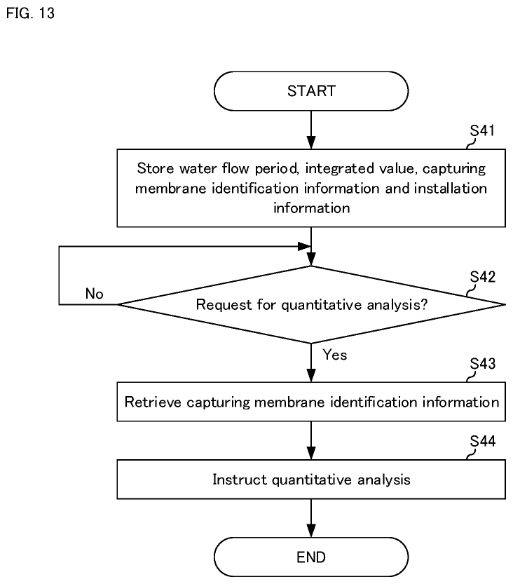

FIG. 13 is a flowchart for explaining an example of the details of the processing of Step S4 described with reference to the sequence diagram shown in FIG. 12. When information is provided from filtration device 101 in Step S3, database 321 stores the period information (water flow period), the integrated value, the capturing membrane identification information, and the installation information that are the provided information in association with each other (Step S41). This association is stored in the form shown in FIGS. 7 and 8.

Thereafter, input unit 311 determines whether or not there is a request for quantitative analysis (Step S42). At this time, input unit 311 may determine that there is a request for quantitative analysis if information corresponding to an operation received from the outside or information transmitted from another device connected to the outside includes a request for quantitative analysis, the installation information, and the date and time information. When there is a request for quantitative analysis, input unit 311 supplies to retrieval unit 331, of the received information, the installation information and the date and time information. Retrieval unit 331 retrieves the capturing membrane identification information from database 321 based on the installation information and the date and time information supplied from input unit 311 (Step S43). Specifically, for example, retrieval unit 331 retrieves the capturing membrane information from database 321 based on the installation information supplied from input unit 311 and retrieves from database 321, of the retrieved capturing membrane information, the capturing membrane identification information that corresponds to the period that includes the date and time information supplied from input unit 311. When the capturing membrane identification information can be retrieved, retrieval unit 331 specifies the retrieved capturing membrane identification information and instructs quantitative device 201 to perform the quantitative analysis (Step S44).

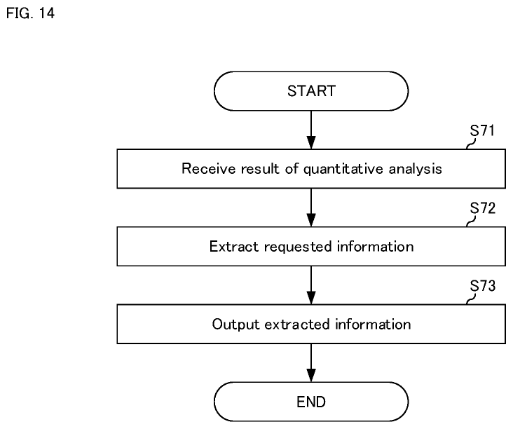

FIG. 14 is a flowchart for explaining an example of the details of the process of Step S8 described with reference to the sequence diagram shown in FIG. 12. When input unit 311 receives the result of the quantitative analysis from quantitative device 201 (Step S71), extraction unit 341 extracts, from the result of the quantitative analysis received by input unit 311, the provided information that is the information corresponding to the input information (Step S72). In some cases, the input information specifies the content of the quantitative analysis (e.g., the type of fine particles to be analyzed). In this case, extraction unit 341 extracts the analysis content contained in the input information from the result of the quantitative analysis performed by quantitative device 201. Subsequently, output unit 351 supplies the provided information extracted by extraction unit 341 (Step S73). Further, quantitative device 201 may calculate the concentration of fine particles in the water to be analyzed using the result of the quantitative analysis and the integrated value measured by the integrating flow meter, so that input unit 311 may receive the concentration of fine particles in the water that is analyzed.

Thus, in the system for performing water quality control, fine particle capturing membranes that are attached to filtration devices are replaced ata predetermined timing. The removed fine particle capturing membranes are stored, and fine particle capturing membranes that were attached to a specified installation location and period are retrieved from among the stored fine particle capturing membranes. Quantitative analyses of the retrieved fine particle capturing membranes are performed and the results are supplied. It is thus possible to perceive the processing status of water that is analyzed at specified locations and specified dates and times.

Although described above by allocating a function (process) to each component, these assignments are not limited to those described above. In addition, as for the configuration of the components, the above-described embodiments are merely examples, and the present invention is not limited thereto. In addition, the present invention can be applied to a system for controlling and managing the content amount of fine particles in a liquid in addition to a system for performing water treatment.

The processing performed by each of the above-described information processing devices 300 and 301 may be performed by logic circuits each manufactured according to the purpose. Further, a computer program (hereinafter, referred to as a “program”) in which the processing contents are described as procedures may be recorded on a recording medium that can be read by information processing devices 300 and 301, and the programs recorded on the recording medium may be read into and executed by information processing device 300 and 301. The recording medium that can be read by information processing devices 300 and 301 refers to a memory or an HDD (Hard Disc Drive) such as ROM (Read Only Memory), RAM (Random Access Memory), or the like incorporated in information processing devices 300 and 301, and further, to a transferable recording medium such as a floppy disk (registered trademark), a magneto-optical disk, a DVD (Digital Versatile Disc), a CD (Compact Disc), a Blu-ray (registered trademark) Disc, and a USB (Universal Serial Bus) memory. The program recorded on the recording medium is read by a CPU provided in each of information processing devices 300 and 301, and the same processing as that described above is performed under the control of the CPU. Here, the CPU operates as a computer that executes a program read from a recording medium on which a program is recorded.

While the present invention has been described with reference to the embodiments, the present invention is not limited to the above embodiments. Various changes that can be understood by those skilled in the art can be made in the configuration and details of the present invention within the scope of the present invention.

This application claims priority based on JP 2020-067756 filed on Apr. 3, 2020 and incorporates all of its disclosure herein.

1. A water quality control method for quantitative analysis and/or qualitative analysis of fine particles contained in water to be analyzed, comprising:
attaching a fine particle capturing membrane for capturing fine particles to a filtration device connected to a flow pipe through which the water to be analyzed flows,
allowing the water to be analyzed to flow from the flow pipe over a predetermined period to the fine particle capturing membrane attached to the filtration device and capturing the fine particles contained in the water to be analyzed to form a fine particle capturing membrane sample, and
performing at an arbitrary timing at least one of quantitative analysis and qualitative analysis of the fine particle capturing membrane sample of a water flow of a period of interest.

2. The water quality management method according to claim 1, wherein after completion of the predetermined period, the fine particle capturing membrane sample is brought into a sealed state, and the fine particle capturing membrane sample is left sealed until analysis of the fine particle capturing membrane sample is performed.

3. The water quality management method according to claim 1, wherein the fine particle capturing membrane sample is continuously obtained over a plurality of periods by collecting the fine particle capturing membrane sample, attaching a new fine particle capturing membrane to the filtration device, and repeating flow of the water to be analyzed through the filtration device.

4. The water quality management method according to claim 3, wherein for each of the plurality of fine particle capturing membrane samples, a water flowing period for the fine particle capturing membrane sample is recorded.

5. The water quality management method according to claim 4, further comprising:
when analysis is required after the water to be analyzed has been used in a manufacturing process of a product, performing at least one of quantitative analysis and qualitative analysis of the fine particle capturing membrane sample of a water flow period that corresponds to the time when the product used the water to be analyzed.

6. The water quality management method according to claim 5, further comprising:
recording the capturing membrane identification information uniquely conferred to the fine particle capturing membrane in association with the water flow period, and
when the analysis is required, performing at least one of quantitative analysis and qualitative analysis of the fine particle capturing membrane sample that was recorded in association with the water flow period that corresponds to a time when the product used the water to be analyzed.

7. The water quality management method according to claim 1, wherein an integrating flow meter is provided downstream side from the filtration device in the flow direction of the water to be analyzed.

8. The water quality management method according to claim 1, wherein the flow pipe is a pipe that branches from an ultrapure water producing apparatus to supply ultrapure water to a point of use or a pipe that branches from the pipe.

9. An information processing device, comprising:
an input unit that receives input information based on an operation accepted from outside,
a database that stores period information, which indicates the time when a fine particle capturing membrane through which water to be analyzed flows for capturing fine particles of the water to be analyzed was attached to a flow pipe through which the water to be analyzed flowed in association with capturing membrane identification information uniquely conferred to the fine particle capturing membrane,
a retrieval unit that retrieves the capturing membrane identification information from the database based on date and time information included in input information the input unit receives, and
an output unit that supplies the capturing membrane identification information retrieved by the retrieval unit.

10. The information processing device according to claim 9, wherein
the input unit receives predetermined information when analysis is required after the water to be analyzed has been used in a manufacturing process of a product.

11. An information processing system, comprising:
a filtration device,
an integrating flow meter,
an analyzer, and
an information processing device; wherein:
the filtration device comprises:
a fine particle capturing membrane that is removably provided from the filtration device and that captures fine particles of the water to be analyzed which flows through the fine particle capturing membrane,
the integrating flow meter is provided on the downstream side of the flow direction of the water to be analyzed of the filtration device and measures the integrated value of the water flow rate of the fine particle capturing membrane,
the information processing device comprises:
an input unit that receives input information based on an operation that is accepted from outside,
a database that stores period information that indicates the time when the fine particle capturing membrane was attached to the flow pipe through which the water to be analyzed flows in association with the capturing membrane identification information uniquely conferred to the fine particle capturing membrane,
a retrieval unit that retrieves the capturing membrane identification information from the database based on date and time information included in input information the input unit receives, and
an output unit that supplies the capturing membrane identification information retrieved by the retrieval unit,
wherein the analyzer performs at least one of a quantitative analysis and a qualitative analysis of the fine particle capturing membrane to which the capturing membrane identification information supplied by the output unit is conferred,
wherein the output unit supplies provided information based on the result of analysis performed by the analyzer.

12. The information processing system according to claim 11, wherein the information processing device, further comprises:
an extraction unit that extracts provided information that is information corresponding to the input information based on the result of the analysis performed by the analyzer with respect to the fine particle capturing membrane that was conferred with capturing membrane identification information that was retrieved by the retrieval unit, wherein
the output unit supplies provided information extracted by the extraction unit.

13. The information processing system according to claim 11, further comprising:
a notification unit that performs predetermined notification when a predetermined period has elapsed from the attachment of the fine particle capturing membrane to the flow pipe, or when an integrated value measured by the integrating flow meter from the attachment of the fine particle capturing membrane to the flow pipe reaches a predetermined value.
